(19) United States
(12) Patent Application Publication
(10) Pub. No.: US 2025/0099403 A1
(43) Pub. Date: Mar. 27, 2025

(54) IDENTIFICATION OF ACTIVITIES OF GUT MICROBE METABOLITES

(71) Applicant: UNIVERSITY OF SOUTH CAROLINA, COLUMBIA, SC (US)

(72) Inventors: JIE LI, COLUMBIA, SC (US); HEXIN CHEN, IRMO, SC (US); DAPING FAN, COLUMBIA, SC (US)

(21) Appl. No.: 18/785,534

(22) Filed: Jul. 26, 2024

Related U.S. Application Data
(60) Provisional application No. 63/565,043, filed on Mar. 14, 2024.
(60) Provisional application No. 63/585,333, filed on Sep. 26, 2023.

(51) Int. Cl. A61K 31/145; A61K 35/74; G16B 30/10; G16H 50/20

(52) U.S. Cl. CPC A61K 31/145; A61K 35/74; G16B 30/10; G16H 50/20

(57) ABSTRACT

Disclosed are treatment methods and compositions developed by the method utilizing Alistipes-derived sulfonolipids in treatment of a variety of diseases, including inflammatory bowel diseases and cancers. Also disclosed are methods for determining a correlation between a gut microbe metabolite and a disease state. The method utilizes a biosynthetic enzyme-guided correlation approach to determine particular metabolites functional in human health as well as a guide for examining the functionality thereof.

### CROSS REFERENCE TO RELATED APPLICATION

The present application claims priority to U.S. Provisional Patent Application Ser. No. 63/585,333, having a filing date of Sep. 26, 2023, and U.S. Provisional Patent Application Ser. No. 63/565,043, having a filing date of Mar. 14, 2024, all of which are incorporated herein by reference in its entirety.

### GOVERNMENT SUPPORT CLAUSE

This invention was made with government support under R35 GM150565 awarded by the National Institutes of Health. The government has certain rights in the invention.

### BACKGROUND

Inflammatory bowel disease (IBD), which includes Crohn's disease and ulcerative colitis, is a chronic condition that affects millions of people worldwide. Unfortunately, there is no effective and safe FDA approved drug for the prevention and treatment of IBD.

Atherosclerosis is the underlying cause of heart attack and stroke. Breast cancer, melanoma and lung cancer are three most common cancers in the US and worldwide. There are many approved drugs for the treatment of atherosclerosis, breast cancer, melanoma and lung cancer. Unfortunately however, due to drug resistance as well as heterogenicity of the pathogenesis, these diseases remain very prevalent and with high mortality.

The gut microbiota plays a key role in health and disease and the trillions of microorganisms inhabiting the human gut are intricately linked to human health. The balance between beneficial genera (such as Alistipes and Odoribacter) and harmful species (such as Escherichia coli) dictates the gut health as well as the overall health of an individual. At the species abundance level, correlational studies have connected specific bacterial taxa to various diseases. However, the underlying mechanisms are largely unknown, and only very limited discoveries in this regard have been reduced to clinical practice. For instance, there is growing evidence that the gut microbiome plays an instructive role in modulating cancer immunotherapy. For example, mice that lack gut microbiota or those pre-treated with antibiotics have a significantly diminished response to immune checkpoint inhibitor (ICI) immunotherapy. A variety of gut microbiota species have been correlated with positive clinical response to immunotherapy and/or shown to be effective in augmenting ICI responses in preclinical cancer models. However, it remains largely unknown how gut microbiota dictate or shape extraintestinal immune responses to modulate tumor behavior.

While the abundances and/or composition of bacteria in the gut serve as good indicators for disease progression, understanding the functional metabolites they produce is critical to decipher how these microbes influence human health. A remarkable class of such functional metabolites is microbe-derived lipids. Biosynthesized de novo by human gut microbiota or biotransformed from dietary nutrients, these lipids can be directly sensed by the host to modulate both metabolic and immunological pathways. Many studies have focused on common microbe-derived lipids, such as short chain fatty acids and phospholipids but there remain a significant number of underexplored lipids which may be equally capable of influencing human health.

While untargeted metabolomic methods have been developed to probe potential functional metabolite trends in disease, these methods face challenges such as the complexity of the metabolome, the lack of reference databases for identification, trace metabolite amounts, and a high degree of variability between different metabolomes, all of which leads to difficulty in revealing specific gut microbial functional metabolites as drivers of molecular mechanisms in disease.

What is needed in the art is a method for determining correlations between gut microbial metabolites and disease states, thereby enabling use of bacterial metabolites for human disease prevention and treatment. Such methods could provide a route for improved therapies for diseases based upon gut microbiota and/or metabolites thereof.

### SUMMARY

According to one embodiment, disclosed is a method for regulating toll-like receptor 4 (TLR4) signaling. The method can include delivering a sulfonolipid (SoL), e.g., a bacterial-derived sulfonolipid, to immune cells, e.g., macrophages. Upon the delivery, TLR4 signaling within the cell can be suppressed.

Also disclosed are methods for treating inflammatory bowel disease (IBD) or a cancer, e.g., breast cancer. A disease treatment method can include administering a microbial SoL to a subject in need thereof, either through direct administration of the SoL or through indirect administration via delivery of a gut microbe that derives the Sol.

Also disclosed is a composition that includes a bacterial sulfonolipid and/or a gut microbe capable of forming the bacterial sulfonolipid in conjunction with a pharmaceutically acceptable carrier.

Also disclosed is a method for determining a correlation between a gut microbial metabolite and a disease. A method can include identifying a reference gene sequence of an enzyme utilized in microbial derivation of the metabolite. Similar enzyme sequences can then be identified in a bacterial reference genome, e.g., enzyme sequences exhibiting a sequence similarity of about 50% or greater. These identified enzyme sequences can be classified according to genera and/or bacterial families. Each classification can then be broken into subfamilies, with each member of a subfamily exhibiting a high sequence similarity to one another, e.g., a sequence similarity of about 90% or greater. A metagenomic analysis of subfamilies can then be carried out to compare the abundance of each examined subfamily in a healthy sample with that of a diseased sample, thereby identifying correlations between the genetic subfamilies and a disease state. Those subfamilies exhibiting a high metagenomic correlation can then be examined according to a metatranscriptomic analysis to compare the abundance of the transcription products of the examined subfamilies in a health sample vs. that of a diseased state. A metabolomic analysis can also be carried out to compare the abundance of the metabolite itself in a healthy sample with that of a diseased sample. Through the multi-level analysis a strong correlation can be determined between the metabolite and the disease.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a flow chart for a method as described herein for correlating a gut microbial metabolite with a disease state.

FIG. 2 presents a pathway of TLR4 activation by lipopolysaccharide (LPS) and interaction of sulfonolipids (SoLs) therewith.

FIG. 3 illustrates immune system modulation schemes of SoLs as may be beneficial in treatment of disease.

FIG. 4 illustrates a flow chart for an exemplary microbial metabolite correlation method as described herein.

FIG. 5 illustrates a phylogenetic tree including 42 species, the genomes of which were found to encode three putative SoL biosynthetic enzymes.

FIG. 6 illustrates the difference in prevalence of Sol biosynthetic enzyme subfamilies between IBD and non-IBD cohorts.

FIG. 7 presents the different expression profiles of 8 SoL biosynthetic enzyme subfamilies in IBD and non-IBD cohorts by prevalence (top) and abundance (bottom).

FIG. 8 presents box plots showing the difference in intensity of SoLs detected in IBD and non-IBD samples.

FIG. 9 provides an histological image analysis of an IBD model both pre- and post-IBD addition of piroxicam.

FIG. 10 graphically presents the histology scores and gross pathology scores of an IBD model with a non-IBD control.

FIG. 11 presents total ion chromatograms obtained from HPLC-HRMS analysis of fecal pellet extracts of IBD model and non-IBD control.

FIG. 12 provides MS/MS spectra of SOL A and SoL B from fecal pellet extracts of an IBD model and non-IBD control.

FIG. 13 provides the difference in expression of SOL A, Sol B, TNFα, IL-6, NOS2, and IL-1β in an IBD model and non-IBD control.

FIG. 14 illustrates the relative expression of TNFα in extract fractions from Alistipes and Odoribacter bacterial strains.

FIG. 15 illustrates a map of bioactivity scores and relative peak areas for several SoL components in an exemplary extract fraction and includes the structures of several different Sol components of the fraction.

FIG. 16 presents relative expression of three inflammatory cytokines (IL-6, TNFα, and IL-1β) by macrophages treated with SoL A with or without LPS and/or Pam3CSK4.

FIG. 17 compares the chemical structures of SOL A and SoL B with those of a sulfatide and the immunogenic portion of an LPS (lipid A).

FIG. 18 illustrates the molecular docking of lipid A, sulfatide, SoL A, and Sol B with MD-2 in complex with TLR-4.

FIG. 19 presents the binding behavior of SOL A and SoL B with MD-2 in competition with biotinylated LPS.

FIG. 20 provides a Western blot analysis of protein levels of IκBα as well as total and phosphorylated ERK1/2 and p38, after treatment with LPS (100 ng/ml) with or without various concentrations of SOL A or B.

FIG. 21 presents the relative cytokine expression of M1 macrophage markers upon polarization with or without the presence of SOL A.

FIG. 22 presents the relative expression of M2 macrophage markers upon polarization with or without the presence of SOL A.

FIG. 23 presents tumor growth kinetics of H605 breast cancer tumor cells in control and mice colonized with A. timonensis.

FIG. 24 presents overall survival of the mice.

FIG. 25 presents quantitative analysis by fluorescence activated cell sorting (FACS) of tumor immune cells of the mice.

FIG. 26 presents FACS analysis of CD86 expression in gated macrophages of the mice.

FIG. 27 presents FACS analysis of IFN-γ and granzyme B (GzmB) expression in gated CD8+T cells of the mice.

FIG. 28 presents serum cytokine expression levels in the mice.

FIG. 29 presents the presence of A. timonensis genomic DNA in different types of samples from the mice.

FIG. 30 presents a TEM image of outer membrane vesicles of an Alistipes culture.

FIG. 31 provides the HPLC-MS analysis of the outer membrane vesicles.

FIG. 32 presents qRT-PCR analysis of cytokine expression in RAW264.7 cells treated with 10UM of SOL A or 100 ng/ml of LPS.

FIG. 33 presents FACS analysis of recruited monocytes (Mono) and neutrophils (Neut) in the peritoneal exudate of mice injected with SoL A.

FIG. 34 presents ELISA analysis of serum IL-6 levels in the mice injected with SOL A.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to methods for determining a correlation between a gut microbe metabolite and a disease state as well as to treatment methods and compositions that have been developed through utilization of the determination method.

The determination method utilizes a biosynthetic enzyme-guided disease correlation approach to uncover microbial functional metabolites as potential molecular mechanisms in human health. The disclosed method can beneficially connect the derivation via biosynthesis or biotransformation of functional metabolites directly to human health conditions. Different from correlating microbial species abundance with disease followed by various efforts to identify functional metabolites, the disclosed approach can streamline this process in a targeted manner by analyzing changes in biosynthetic enzyme expression patterns in response to disease. The approach utilizes knowledge of biosynthetic enzymes that are required for the biosynthesis of corresponding functional metabolites. Through determination of increased or decreased expression of these enzymes, the change in production of a specific functional metabolite can be correlated with different disease parameters to reveal positive or negative associations. This process can enable a more focused, targeted metabolomic analysis to rapidly filter metabolites of interest and confirm or disprove their association with a disease.

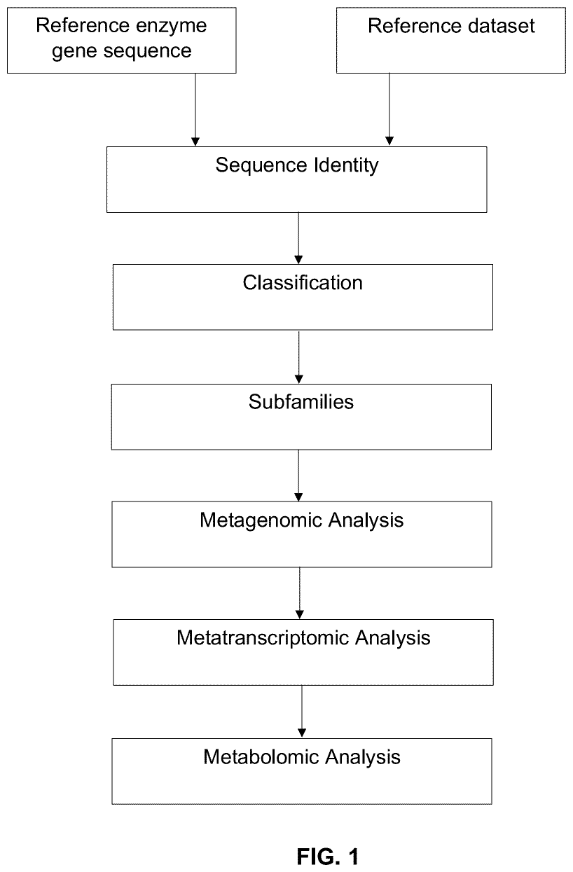

FIG. 1 presents one embodiment of a general flow chart for a method. As indicated, a method can include identifying a reference gene sequence for at least one enzyme that is involved in the microbial formation of a metabolite of interest. While the disclosed method can be carried out with only a single enzyme, the methods are not limited to such, and in other embodiments, a method can utilize more than one enzyme necessary for the production of the metabolite of interest, e.g., two, three, or more enzymes utilized in microbial production of a metabolite of interest.

The reference enzyme genetic sequence(s) can then be compared to a reference genome dataset. Due to the widespread availability of large high-quality sequencing datasets, disclosed methods can provide a large number of potential correlating sequences, which can then lead to high confidence determination of a tie between the metabolite of interest and a disease state.

A reference data set can include any data set that includes multiple gut bacterial genomes. A reference data set can include single amplified genomes (SAGs), metagenome-assembled genomes (MAGs), or any combination thereof. In one embodiment, a reference data set can include genomes of the catalog of the human gut microbiome described in Nat Biotechnol 39, 105-114 (2021). Other reference genomes as are known in the art may alternatively or additionally be utilized, such as, and without limitation to, genome catalogues as described by Li et al. (npj Biofilms and Microbiomes 9, 45 (2023)), Zou et al. (Nature Biotechnology 37, 179-185 (2019)), Mukherjee et al. (Nature Biotechnology 35, 676-683 (2017)), the European Nucleotide Archive (ENA) assemblies under the Umbrella project PRJEB46036, the Compact Bit-sliced Signature (COBS) index, the minHash index, the pp_sketch index, etc.

A comparison between the reference enzyme sequence and the reference dataset can be utilized to develop a set of homologous enzyme sequences from the reference dataset. In general, a homologous enzyme sequence of the reference dataset can be one that exhibits a sequence similarity of about 50% or greater to the reference genetic sequence. As utilized herein, the “sequence similarity” can be determined according to the following parameters:
1. E-value, which is computed according to the gap-corrected Karlin-Altschul statistics.
2. Coverage as defined by the number of aligned residue pairs divided by the maximum length of the compared sequences.
3. Sequence identity is defined as the number of identical aligned residues divided by the number of aligned columns.

In some embodiments, UBLAST or mmseqs2 can be used to identify homologous enzymes of the predetermined sequence similarity.

In one embodiment, a determination of homology can include one or more additional parameters, in addition to a predetermined sequence similarity. For instance, in one embodiment, in addition to predetermined level of sequence similarity, homologous regions of the similar sequences can include similar protein domains as determined by a Pfam domain analysis (Bateman et al. (Nucleic Acids Res. 2000 Jan. 1; 28 (1): 263-266). Similar protein domains can generally be defined as those containing corresponding Pfam domains having a hit score of about 50 or greater as determined by use of, e.g., hmmsearch (HMMER v. 3.3).

In those embodiments in which more than one reference enzyme sequence is examined, the set of homologous enzymes can be further narrowed in one embodiment to those of genomes that include all of the examined enzymes.

Following determination of a set of homologous enzyme sequences, the identified sequences can be classified, e.g., by phylum, genera, and species. Depending upon the resulting classifications, some of the classes thus identified may be eliminated from further examination. For instance, homologous enzymes of a classification belonging to a non-gut bacterial genus or a phylum, genus, or species that is known to be otherwise not feasible for further examination (e.g., toxicity, etc.) can be eliminated from further examination in some embodiments.

Following classification, the members within each class of interest can be further separated into subfamilies, with members of each subfamily having a high sequence similarity to one another, e.g., about 90% or greater sequence similarity as determined by UBLAST or mmseqs2. Further, the subfamilies can be prioritized, e.g., those subfamilies in which the members have a very high sequence similarity, e.g., about 95% or greater being prioritized over those in which the members have a relatively lower sequence similarity, e.g., from about 90% to about 95%.

While each member of each of the subfamilies can be further examined in one embodiment, in other embodiments, only a representative sample of the members (e.g., from one to about 10 members) can be selected for further examination. For example, in one embodiment a single genome of each subfamily can be selected from each prioritized subfamily for further examination.

Upon determination of the subfamilies and optional selection of a representative number of genomes therefrom for further examination, the examined subfamilies or representative genome(s) thereof can be the basis for analysis via metagenomics. To carry out a metagenomics analyses, datasets for healthy subjects and for subjects identified as having the disease state of interest can be obtained for comparison from sources as are known in the art, e.g., the NCBI Sequence Read Archive (SRA), or the like. For instance, when examining a disease state that includes IBD, a metagenomic dataset can be obtained from the Inflammatory Bowel Disease Multi-omics Database. Similarly, other datasets as are known and available in the art can be utilized for other diseases.

When carrying out a metagenomic analysis, the representative gene sequence(s) of the metabolite enzyme(s) determined from a selection protocol as described above can be compared to the metagenomic datasets of the healthy and diseased cohorts. This comparison can reveal a statistically significant difference (e.g., Jaccard distance, PERMANOVA p=0.001; Fisher's exact test p≤0.05; and/or a difference in prevalence of ≥10%) in the presence of the genetic sequence of the enzyme between the healthy and diseased cohorts and accordingly a statistically significant difference in the gut microbial capacity for producing the metabolite of interest between healthy individuals and those identified with the disease state of interest.

To further elucidate a tie between the metabolite of interest and the disease state, a metatranscriptomic analysis of the enzyme(s) can be carried out. Healthy and disease transcriptome datasets can generally be obtained from the same source as the metagenomic datasets. However, in some embodiments, a different dataset source may be utilized, generally depending upon the particular disease state of interest and the publicly available datasets therefore. Comparative analysis between the enzyme transcript(s) of the prioritized subfamilies developed as described and the healthy and diseased transcriptome datasets can delineate that not only capacity for producing the enzyme(s), but expression of the enzyme(s) is also significantly different (e.g., Bray-Curtis distance, PERMANOVA p=0.001; Fisher's exact test p≤0.05; and/or a difference in prevalence of ≥10%) between healthy and disease cohorts.

Metabolomic analysis of the metabolite produced by the examined enzyme(s) can also be used in some embodiments to further elucidate the tie between the metabolite and the disease state. Healthy and disease metabolome datasets for comparison can generally be obtained from the same source as the metagenomic and/or metatransciptomic datasets used for the metabolite enzyme. However, in some embodiments, a different dataset source may be utilized, generally depending upon the particular disease state of interest and the publicly available datasets therefore. In carrying out a metabolomic analysis, an analysis can be carried out based upon a generic classification of the metabolite, e.g., sulfonolipids in general. Additionally or alternatively, a metabolite analysis can be carried out on one or more particular species of the metabolite, so as to further differentiate activities of the different metabolites in a diseased state. For instance, when considering the general classification of sulfonolipid bacterial metabolites, there are multiple different specific sulfonolipids, e.g., SoL A, Sol B, SoL C, etc., that can exhibit differences in prevalence in a disease state as well as in functionality. Accordingly, in some embodiments, metabolite features can be assigned to each species of the metabolite, and then the healthy and disease cohorts can be examined to determine statistically significant differences in prevalence between the two for each of the species.

Through the multi-level analysis techniques, a high confidence can be obtained in determining a correlation between a gut microbe metabolite and a disease state. This information can then be utilized in developing therapeutic treatments and compositions for use in treating the disease state. For instance, Alistipes and Odoribacter are two commensal genera of the human gut microbiome with emerging significance in modulating health, particularly in the two primary forms of inflammatory bowel disease (IBD): Ulcerative colitis (UC) and Crohn's disease (CD). Species abundances from both genera have been found to negatively correlate with IBD pathogenesis, suggesting that they may play a protective role against IBD. Additionally, some species have been shown to ameliorate symptoms of IBD. However, knowledge of specific metabolites of these genera as well as functionality of their metabolites has not been developed.

Both Alistipes and Odoribacter are prolific SoL producers. Through application of analysis techniques as described herein, the expression of gut microbial SoL bioderived enzymes has been correlated with inflammatory bowel disease (IBD) in patients, revealing a negative correlation. As described further below, this correlation has been further corroborated by targeted metabolomic analysis, identifying that abundance of SoLs is significantly decreased in IBD patient samples. This finding has been validated in a mouse model of IBD described further herein, showing that SoL production is indeed decreased while inflammatory markers are increased in mice with IBD.

Identification of a tie between IBD and SoLs has led to further understanding of the activity of this particular gut microbe metabolite and through that understanding, development of treatment methods and compositions for treatment of IBD as well as other diseases.

More specifically, disclosed herein are methods for modulating TLR4 signaling in an immune cell through administration of a Sol to the cell. While the causes of IBD remain largely unknown, IBD progression has been linked to aberrant TLR signaling. TLRs are pattern recognition receptors that initiate a variety of host processes, especially inflammatory responses, through the recognition of pathogen-associated molecular patterns (PAMPs) and other non-pathogenic microbial factors. Specifically, TLR2 and TLR4 are well-known to recognize PAMPs in the gut microbiome. In addition, their expression is significantly increased in IBD pathogenesis, reflecting a state of aberrant activation.

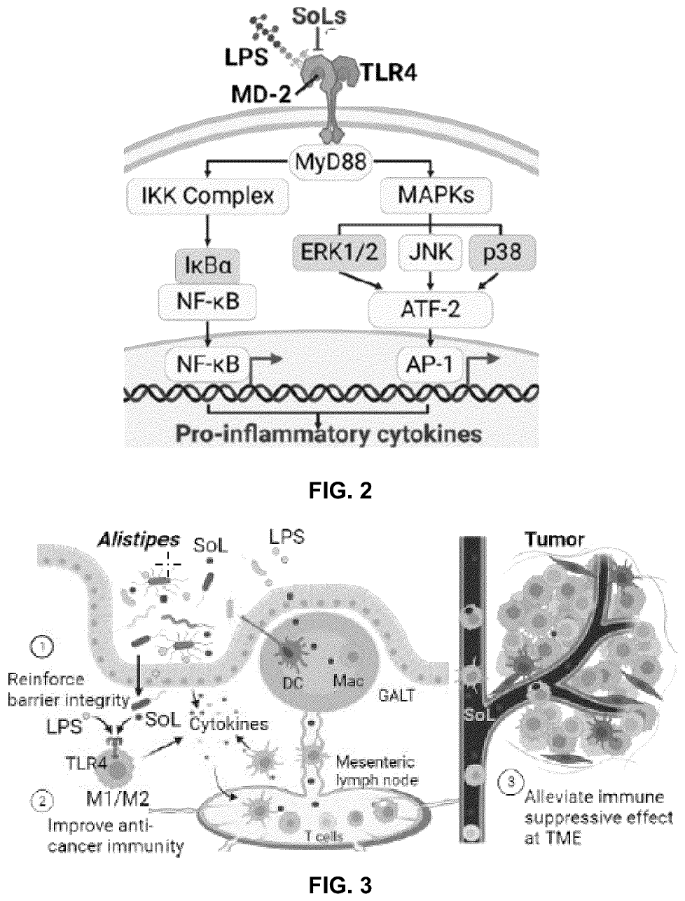

Without wishing to be bound to any particular theory, FIG. 2 schematically illustrates the role SoLs are understood to play in modulating TLR4 signaling. Upon successful binding with TLR4's natural ligand lipopolysaccharide (LPS) to myeloid differentiation factor 2 (MD-2), TLR4 initiates downstream signaling, such as through the NF-κB and MAPK pathways, resulting in the induction of inflammatory cytokine expression. As illustrated in FIG. 2, SoL can mediate immunomodulatory activity of TLR4 through blocking TLR4's LPS binding to MD-2, and can even displace LPS from MD-2 at higher concentrations, leading to suppression of TLR4 signaling pathways and macrophage M1 polarization. Upon SoL binding to MD-2 in place of LPS, activation of the TLR4 pathway can be blocked, effecting the phosphorylation of TLR4-downstream signaling molecules, ERK1/2 and p38, and the degradation of IκBα, which are critical for LPS-induced cytokine expression and resulting inflammation.

As illustrated in FIG. 3, SoLs released from a gut microbe such as an Alistipes through outer-membrane vesicles can play a dual immunoregulatory role in a spatial manner: In the gut, a Sol can taper microbial LPS-induced inflammation and collateral damages via competitively binding to TLR4 as illustrated in FIG. 2 and thereby can reinforce the barrier, which can be highly beneficial in treatment of IBD and related diseases.

In addition, however, in the circulation system and a tumor environment, SoL can enhance anti-cancer immunity via activating innate immune cells and alleviating immune suppression in the tumor micro-environment (TME), for instance through activation of innate cells in lymphoid organs through binding to TLR4 as well as by inducing inflammation and alleviating immune suppressiveness in TME.

Accordingly, in one embodiment, mediation of TLR4 immunomodulatory activity through administration of a Sol can be utilized in treatment of IBD. However, disclosed TLR4 modulation methods can be applicable in other disease treatment methods as well including, without limitation, inflammatory bowel disease, atherosclerosis, breast cancer, melanoma, and lung cancer. For example, and as described in more detail below, Alistipes colonization can inhibit tumor growth and can enhance the anti-tumor immune responses.

In one embodiment, treatment methods and compositions can utilize one or more SoLs derived from Alistipes. The genus Alistipes includes at least 13 species: Alistipes finegoldii, Alistipes putredinis, Alistipes onderdonkii, Alistipes shahii, Alistipes indistinctus, Alistipes senegalensis, Alistipes timonensis, Alistipes obesi, Alistipes ihumii, Alistipes inops, Alistipes megaguti, Alistipes provencensis, and Alistipes massiliensis. Alistipes communis and A. dispar, and the subspecies A. Onderdonkii subspecies vulgaris (vs. onderdonkii subsp.) are the newest strains featured outside that list. In one embodiment, a treatment method and composition can include a Sol e.g., sulfobacin A (SoL A), derived from a beneficial human gut Alistipes gut microbe, as such microbes and SoLs are safe for humans. For instance, a treatment method or composition can include a Sol derived from Alistipes timonensis (A. Tim), Alistipes finegoldii (A. fin), or Alistipes putredinis (A. put).

Beneficially, the bacteria can be cultured in large scale according to standard culturing practices, and as such the cost can be relatively low, particularly when compared with synthesized chemical drugs or more complicated protein drugs. Upon culturing, a composition formation method can include isolating and purifying one or more SoL metabolites from the bacterial culture.

A pharmaceutical composition can include one or more SoLs in a pharmaceutically effective amount in conjunction with a pharmaceutically acceptable carrier. In one embodiment, a pharmaceutical composition can include one or more SoLs in conjunction with one or more species of the genera Alistipes.

The term “pharmaceutically effective amount” refers to the amount of a drug or pharmaceutical agent that will elicit the biological or medical response of a tissue, system, animal, or human that is being sought by a researcher or clinician. This amount can be a therapeutically effective amount.

The term “pharmaceutically acceptable carrier” is used herein to refer to a carrier that is useful in preparing a pharmaceutical composition that is generally safe, non-toxic, and neither biologically nor otherwise-undesirable, and is acceptable for veterinary use as well as human pharmaceutical use. A “pharmaceutically acceptable carrier” can include both one and more than one such carrier. By “pharmaceutically acceptable,” it is meant that the carrier is compatible with the other ingredients of the formulation and not deleterious to the recipient thereof.

Pharmaceutical compositions may be prepared by any of the methods well known in the art of pharmacy. Pharmaceutical compositions encompass any compositions made by admixing the active ingredients and a pharmaceutically acceptable carrier. The carrier may take a wide variety of forms depending on the form of preparation desired for administration, e.g., oral or parenteral (including intravenous), as well as the presence or absence of Alistipes.

The terms “administration of” or “administering a” pharmaceutical composition should be understood to mean providing a pharmaceutical composition to an individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal deliveries.

Thus, the pharmaceutical composition can be formed as discrete units suitable for oral administration such as capsules, cachets, or tablets, each containing a predetermined amount of the active ingredients. Further, the composition can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the composition may also be administered by controlled release means and/or delivery devices. The foregoing list is illustrative only and is not intended to be limiting in any way.

Pharmaceutical compositions intended for oral use may contain one or more agents selected from the group consisting of sweetening agents, flavoring agents, coloring agents, and preserving agents in order to provide pharmaceutically elegant and palatable preparations. Tablets may contain a composition having at least one of the compounds described herein in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid, or talc. The tablets may be uncoated or they may be coated by known techniques to delay disintegration and absorption in the gastrointestinal tract and thereby provide a sustained action over a longer period. A tablet may be prepared by compression or molding, optionally with one or more accessory ingredients or adjuvants. Compressed tablets may be prepared by compressing, in a suitable machine, at least one of disclosed compounds in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions for oral use may also be presented as hard gelatin capsules wherein one or more of the disclosed compounds is mixed with an inert solid diluent, for example, calcium carbonate, calcium phosphate or kaolin, or as soft gelatin capsules wherein the compound(s) is/are mixed with water or an oil medium, for example peanut oil, liquid paraffin, or olive oil.

Pharmaceutical compositions can also include aqueous suspensions, which contain the active materials in admixture with excipients suitable for the manufacture of aqueous suspensions. In addition, oily suspensions may be formulated by suspending at least one of the disclosed compounds in a vegetable oil, e.g., arachis oil, olive oil, sesame oil or coconut oil, or in a mineral oil such as liquid paraffin. Oily suspensions may also contain various excipients. The pharmaceutical composition may also be in the form of oil-in-water emulsions, which may also contain excipients such as sweetening and flavoring agents.

Pharmaceutical compositions can be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and should be preserved against the contaminating action of microorganisms such as bacteria and fungi.

Pharmaceutical compositions can also be in a form suitable for rectal administration, wherein the carrier is a solid. Suitable carriers include cocoa butter and other materials commonly used in the art.

In those embodiments in which the composition can include an Alistipes microorganism in conjunction with the Sol metabolite of the Alistipes, the composition may be supplemented with nutrients suited for the maintenance (or growth) of the microorganism. The nutrients can, for example, include nutrient media for culture of the microorganism. Examples of the nutrients can include various oligosaccharides, such as lactooligosaccharide, soy oligosaccharide, lactulose, lactitol, fructooligosaccharide, and galactooligosaccharide. The amount of such nutrients is not particularly restricted but is generally in a range of about 1 wt. % to ab out 3 wt. %.

A composition may be supplemented with suitable amounts of various vitamins, trace metal elements, and so forth, as may be utilized by the Alistipes microorganism or the subject. Examples of vitamins can include, without limitation, vitamin B, vitamin D, vitamin C, vitamin E, and vitamin K. Examples of trace metal elements are zinc, selenium, iron, manganese, etc.

The quantity of an Alistipes microorganism to be formulated in a composition can in one embodiment be from about 10⁷ colony forming units (cfu) of Alistipes bacteria or more, for example at least about 10⁷ cfu, at least about 10⁸ cfu, at least about 10⁹ cfu, at least about 10¹⁰ cfu, at least about 10¹¹ cfu, or at least about 10¹² cfu.

The present invention may be better understood with reference to the examples set forth below.

### Example 1

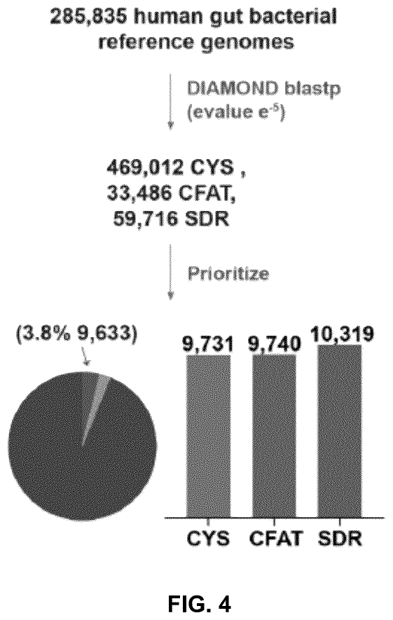

A systematic investigation of the biosynthetic potential of SoLs was carried out utilizing 285,835 human gut bacterial reference genomes including single amplified genomes (SAGs) and metagenome-assembled genomes (MAGs) (Almeida, A. et al. A unified catalog of 204,938 reference genomes from the human gut microbiome. Nat Biotechnol 39, 105-114 (2021)). As illustrated in FIG. 4 based on sequence homology with experimentally verified SoL biosynthetic enzymes, a total of 562,214 homologous enzyme sequences were identified, including 469,012 cysteate synthases (CYS), 33,486 cysteate fatty acyltransferases (CFAT), and 59,716 short-chain dehydrogenases/reductases (SDR). The 562,214 putative SoL biosynthetic enzymes were identified across 21 bacterial phyla.

Upon examining phylogenetic trends, it was found that these three enzymes were widely distributed in 255,572 genomes across 21 phyla, with the majority belonging to Bacteroidota and Firmicutes. A subset of 6.21% (15,863/255,572) of the genomes was found to encode all three putative SoL biosynthetic enzymes. To prioritize them for further analysis, the homologs were filtered on the basis of three rules:
(1) the homology of both CFAT and CYS must equal or exceed 50% sequence similarity with experimentally validated CFATs and CYSs, as these enzymes are the first two specific enzymes in the biosynthetic pathway of SoLs that distinguish the biosynthesis between SoLs and SLs;
(2) the homologous regions of CYS, CFAT, and SDR should include protein domains with Pfam IDs PF00291, PF00155, and PF00106, respectively (hit score >50);
(3) a set of homologous enzymes, especially SDR enzymes that show variable sequence similarities, should come from the same genome encoding all three enzymes as all three are required for SoL biosynthesis, thus ensuring co-occurrence.

Applying these rules, 9,731 CYS (1,384 unique sequences), 9,740 CFAT (917 unique sequences), and 10,319 SDR enzymes (1,076 unique sequences) were prioritized from 9,633 bacterial genomes (FIG. 4, bar chart showing the number of prioritized SoL biosynthetic enzymes encoded by 9,633 genomes of the pie chart).

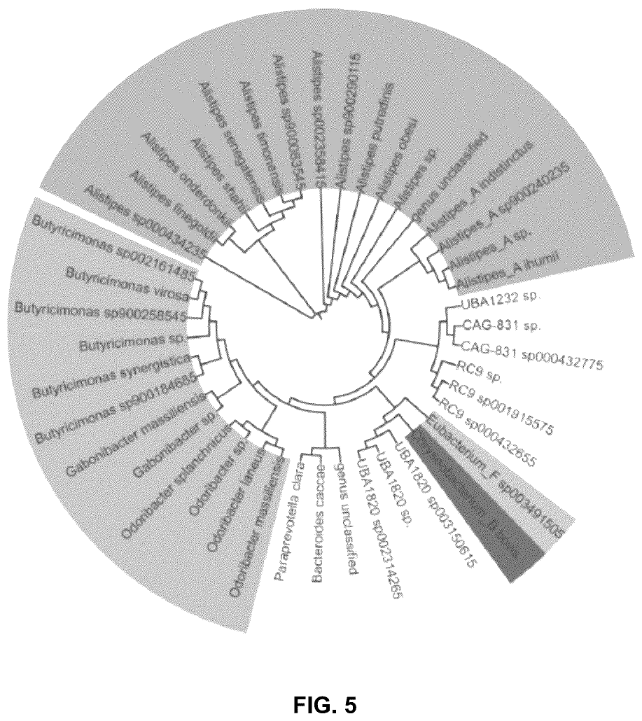

The prioritized enzymes were distributed among 42 species from Bacteroidota and one species from Firmicutes. FIG. 5 presents the results in a circular phylogenetic tree showing the prioritized SoL biosynthetic enzymes found primarily in species from Bacteroidota. The tree is annotated with species names and shaded by taxonomic families, including Rikenellaceae, Marinifilaceae, Weeksellaceae, and Lachnospiraceae. Of note, among the 42 species from Bacteroidota, 71% (30/42) belong to bacterial families that have been previously reported to produce SoLs including Rikenellaceae (containing genera Alistipes and Alistipes_A), Marinifilaceae (containing genus Odoribacter), and Weeksellaceae (containing genus Chryseobacterium B).

To determine a link between gut microbial capacity of producing SoLs and IBD incidence, a comparative analysis of metagenomic and metatranscriptomic data obtained from the Inflammatory Bowel Disease Multi-omics Database (IBDMDB) was conducted.

Sequence similarity networks were generated with a 90% sequence identity threshold to group enzymes with similar functions. Following, the prioritized biosynthetic enzymes were categorized into 214 subfamilies (79 CYS subfamilies; 25 CFAT subfamilies; and 110 SDR subfamilies) for the subsequent analyses.

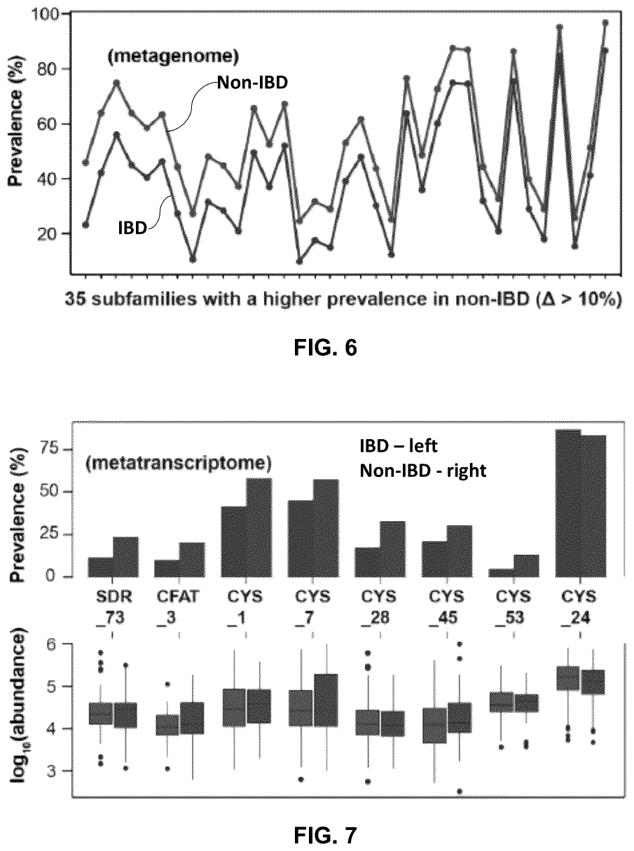

Looking for the presence of the prioritized 214 subfamilies in IBD cohorts, 154 subfamilies were identified in 667 metagenome samples (182 healthy samples and 485 IBD disease samples), of which 116 subfamilies were detected in ≥5% of samples. Beta diversity of the presence of these 116 subfamily biosynthetic enzymes indicated that the overall composition of Sol biosynthetic enzyme subfamilies was significantly different between the healthy and IBD cohorts (Jaccard distance, PERMANOVA p=0.001). A Principal Coordinate Analysis (PCoA) showed differences in the presence profile of overall Sol biosynthetic enzyme subfamilies between IBD and non-IBD groups based on Jaccard distance. Of note, 57 subfamilies had a significantly higher prevalence (Fisher's exact test p<0.05) in healthy individuals as compared to IBD cases, among which 35 subfamilies (18 CYS subfamilies, 2 CFAT subfamilies, and 15 SDR subfamilies) further show a difference of prevalence >10% (FIG. 6) (two-sided Fisher's exact test, p<0.05) with difference of prevalence >10%.

To further examine the difference between the expression profiles of SOL biosynthetic enzymes between the IBD and healthy groups, the comparative analysis was extended to the metatranscriptomic level. It was found that 132 SoL biosynthetic enzyme subfamilies were expressed in 777 metatranscriptomic samples (193 healthy samples and 584 IBD disease samples), with about 42% (55/132) detected in at least 5% of samples. Beta diversity of the expression profiles of Sol biosynthetic enzymes suggested that the overall expression of these enzyme subfamilies is significantly different between the healthy and IBD cohorts (Bray-Curtis distance, PERMANOVA p=0.001).

To capture more detail, the prevalence and abundance differences of each enzyme subfamily in the metatranscriptomic samples was compared. Results are shown in FIG. 7. Nine subfamilies had higher prevalence (Fisher's exact test p<0.05, varying from 9% ˜ 17%) in the non-IBD group than the IBD group. Further, 8 subfamilies were identified (6 CYS, 1 CFAT, and 1 SDR) as significantly different in abundance (expression) profiles between the healthy controls and IBD cases. Notably, 7 of the 8 subfamilies had a higher prevalence. The upper panel of FIG. 7 provides results as bar charts showing the prevalence of the differential Sol biosynthetic enzyme subfamilies across non-IBD and IBD individuals. The lower panel of FIG. 7 provides the one-sided Mann-Whitney U test abundance results (adjusted p-value <0.05). Except for the CYS subfamily24 (no significance), all were significantly higher in prevalence in non-IBD than IBD groups (p<0.05).

Metabolomic evidence was explored in the differences of detectable SoLs, among IBD and non-IBD groups from publicly accessible metabolomics datasets. It was expected that the increased expression of Sol biosynthetic enzymes would correspond with increased production of SoLs in non-IBD groups compared to IBD groups. Using metabolomics data from IBDMDB first, metabolite features were assigned to one of the sulfonolipids: sulfobacin A (SOL A), B (SOL B), C (SoL C), and F (SoL F) by exact mass comparison with mass error less than 5 ppm. As illustrated in FIG. 8, it was found that SoLs A, B, C, and F had significantly higher abundance in non-IBD groups than IBD groups (significance was determined by Wilcoxon rank sum test: * 0.01<p<0.05, ** 0.001<p<0.01, and *** p<0.001). Furthermore, higher SoL abundance was observed in non-IBD samples compared to both IBD subtypes: Ulcerative colitis (UC) and Crohn's disease (CD).

In another independent metabolomics dataset, it was found that the abundance of SOL B was also lower in IBD groups, consistent with the result generated from the first dataset (Wilcoxon rank sum test, p<0.001).

Overall, the metagenomic analysis reflected that SoL biosynthetic enzymes were more prevalent in the non-IBD group than the IBD group, metatranscriptomics suggested that these enzymes are more actively transcribed in the non-IBD group, and metabolomics indicated that representative SoLs are produced in higher abundance in the non-IBD group. Altogether, the findings established a negative correlation directly between SoLs biosynthesis and IBD.

### Methods

Experimentally validated enzymes involved in SoL biosynthesis were collected as reference amino acid sequences of CYS, CFAT, and SDR. Reference amino acid sequences were used as seed sequences to search for homologs in the human gut bacterial reference genomes using the DIAMOND blastp model with an e-value threshold of 10-5. The taxonomic distribution of the resulting homolog sets were then investigated based on the taxonomy annotation for each genome. To prioritize SoL biosynthetic enzymes, the homologs of CFATs and CYSs from genomes which encode copies of CFAT, CYS and SDR were first used to generate sequence similarity networks with experimentally validated CFAT and CYS at a threshold of 50% similarity using MMseqs2. The prioritized CFATs and CYSs meet co-occurrence with SDRs in the same genome. The filtered enzymes were further subjected to Pfam domain analysis by hmmsearch (HMMER v3.3) with default parameters against the Pfam-A database (v33.1). Enzymes containing corresponding Pfam domains with hit score >50 were selected as prioritized SoL biosynthetic enzymes. Prioritized enzymes were used to generate CYS, CFAT, and SDR subfamilies using sequence similarity networks with at least 90% similarity by MMseqs2 clustering. Maximum-likelihood trees were generated using the representative genome of each species by GTDB-Tk (v2). Finally, a representative genome was selected for each species to display the SoL biosynthetic potential and annotate the phylogenetic trends using iTOL.

### Quantification of Enzyme Abundances in Metagenomic and Metatranscriptomic Samples.

Metagenomic and metatranscriptomic whole-genome sequencing datasets of human gut microbiomes related to IBD were downloaded from the NCBI Sequence Read Archive (SRA) (SRA Accessions: PRJNA398089 and PRJNA389280). For both metagenomic and metatranscriptomic samples, reads were quality filtered and adapter removed using bbduk.sh with the following parameters: qtrim=rl ktrim=r mink=11 trimq=10 minlen=40 (read quality cutoff is 10, read length cutoff is 40). High-quality reads were mapped to the nucleotide sequences of corresponding contigs containing the Sol biosynthetic genes using BWA mem algorithm with default parameters. The reads mapped to genes were counted by featurecounts with the following parameters:-f-t CDS-M-O-g transcript_id-F GTF-s 0-p-fracOverlap 0.25-Q 10-primary. Enzymes encoded or expressed in at least 5% of samples were considered as common distribution in humans and were included in the comparative analysis. Transcripts per million (TPM) were calculated for each Sol biosynthetic enzyme gene. The abundance of each cluster was calculated by the sum of the relative abundances of all genes in the cluster. Beta diversity was performed to quantify the prevalence and relative abundance differences in the overall composition of SOL biosynthetic enzymes between the IBD and the control groups. PERMANOVA was performed to show the encoding and expression profile differences of SOL biosynthetic enzymes between IBD and control groups. Both beta diversity and PERMANOVA were performed using the R package vegan. To explore the differences between IBD and control groups of single SoL biosynthetic enzyme subfamilies, the Shapiro-Wilk test was used to evaluate the normality of a specific gene cluster's relative abundance. The significance of relative abundance between healthy and IBD individuals was then calculated using either two-sample Student's t-test (for normally distributed data) or two-sample Wilcoxon rank sum test (for not normally distributed data). Significance tests were performed in Python using packages Pandas and SciPy. The Benjamini-Hochberg method was used to adjust p-values to correct for multiple testing. SoL biosynthetic enzyme subfamilies were considered differential if the adjusted p-value was less than 0.05. For differential Sol biosynthetic enzymes, a two-sided Fisher's exact test was performed to explore their difference in prevalence across IBD and non-IBD groups.

### Analysis of Publicly Available Metabolomics Datasets.

Two publicly available metabolomics datasets were downloaded from IBDMDB and Metabolomics Workbench. Processed metabolomic feature tables were used to search for SoL related metabolites (SoLs A-F). Search parameters were set to the exact mass of SoLs A-F using a 5 ppm match tolerance for parent ions under negative mode. The absolute intensity of relative abundance from resulting matches were used to calculate differential abundances between non-IBD and IBD samples. The Wilcoxon rank sum test was used to measure statistical significance.

### Example 2

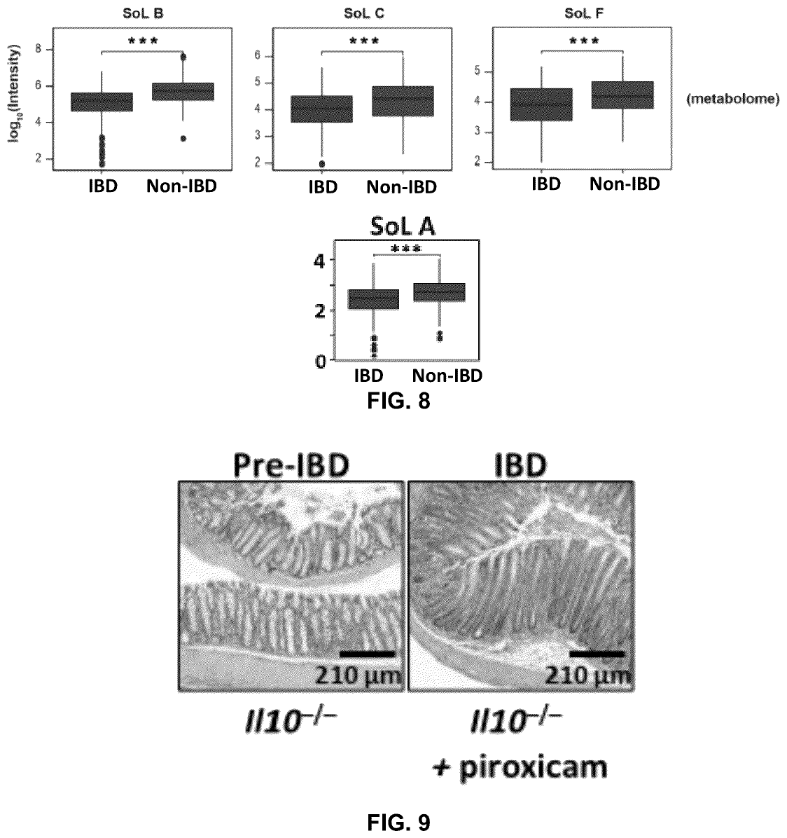

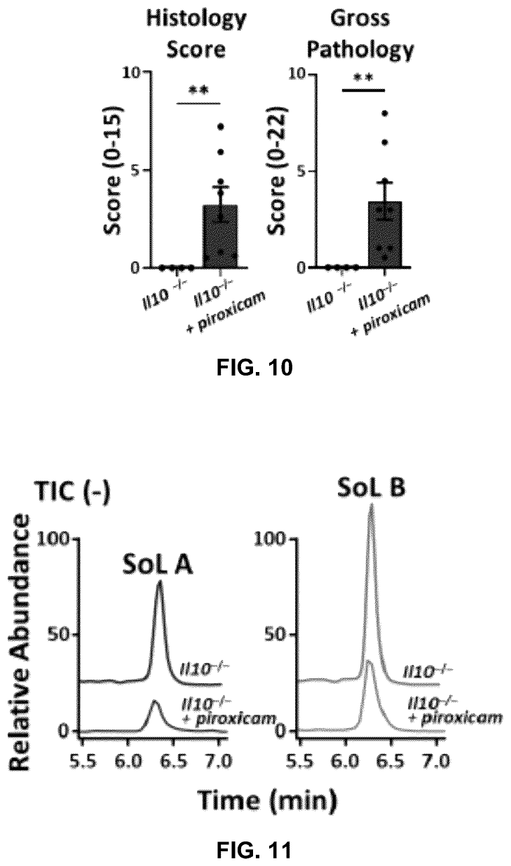

Experimental validation of the predictions of Example 1 were carried out using a mouse model of IBD. A well-established model was used of 1/10-deficient (1/10−/−) mice treated with the non-steroidal anti-inflammatory drug piroxicam, which has been shown to accelerate development of colitis through the disruption of the gut mucosal barrier. Stimulation of mucosal Toll-like receptors (TLRs) stemming from this mucosal barrier breakdown was another factor in the selection of this model, as it has been previously shown that SoL A suppresses LPS-induced inflammation and LPS is well-known to activate TLR signaling. Accordingly, the piroxicam/1/10−/−IBD model was successfully established and it was observed that the colonic tissues were inflamed in the piroxicam-treated (IBD) group when compared to the control (pre-IBD) group as indicated by marked crypt hyperplasia, loss of goblet cells, submucosal edema, and immune cell infiltration into the lamina propria. Gross pathology analysis included qualitative evaluations of cecal atrophy, thickening of cecal and colon tissues, extent of content loss in the cecum, and diarrhea supported induction of IBD with piroxicam treatment. Histological analysis (FIG. 9) of the mouse distal colon revealed that piroxicam treatment induced intestinal inflammation. As indicated in FIG. 10, histology and gross pathology scores indicate induction of IBD in 1/10−/−mice treated with piroxicam (n=7, female) compared to pre-IBD control//10-252/-mice (n=4, female), confirming the successful establishment of IBD model. The trends were consistent in male mice in another independent experiment using the same IBD model.

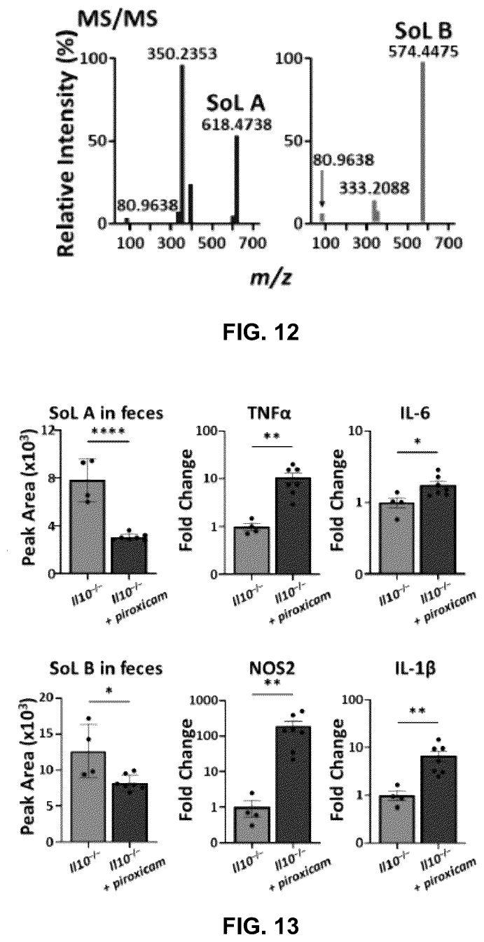

Fecal material was collected from IBD 1/10−/−+piroxicam (n=7, female) and pre-IBD control//10−/−mice (n=4, female), extracted metabolites, and the abundance of SoLs was measured by targeted metabolomics using high performance liquid chromatography (HPLC)-high resolution mass spectrometry (HRMS). Results are shown in FIG. 11, which include total ion chromatograms (TICs) obtained from HPLC-HRMS analysis of the fecal pellet extracts and reveal the presence of SOL A and SoL B. SoL abundances appear to be decreased in 1/10−/−+piroxicam mice fecal pellets. Metabolites with m/z corresponding to major SoLs were detected, specifically SoLs A and B, in all samples tested and their identities were unambiguously determined by HPLC-MS/MS. As indicated (FIG. 12), the MS/MS spectra confirm the identities of Sol A and SoL B based on the presence of the 80 m/z fragment characteristic of sulfonate-containing compounds as well as when compared to literature fragmentation patterns.

It was then determined that the abundances of both SoLs A and B in feces were significantly decreased in piroxicam treated samples compared to 1/10−/−control samples (FIG. 13). Peak areas were calculated using TICs obtained after MS/MS fragmentation and used to measure the abundance of SoLs A and B. Significance was determined using Student's t-test. Expression of inflammatory markers TNFα, NOS2, IL-6, and IL-1β were significantly increased in 1/10−/−+piroxicam mice (FIG. 13). Significance was determined using Mann-Whitney U test. Bars represent mean±standard error. For all p values: ** 0.001<p<0.01 and **** p<0.0001.

The results confirmed the informatic analysis and directly established a negative correlation between SoLs production and IBD progression in the mouse model. In addition, significantly increased expression of the NF-κB-regulated inflammatory markers TNFα, NOS2, IL-6, and IL-1β was also observed in the IBD mouse group, further indicating a negative correlation between Sol production and these inflammatory markers. To eliminate any differences caused by sex, another independent study was performed with male mice using the same IBD model and the same negative correlation was observed between SoLs production and IBD progression.

### Methods

Animals. 1/10−/−mice were purchased and maintained in specific pathogen free conditions on a 12-hour light/dark cycle with unlimited access to water and food (Envigo 8904).

Piroxicam-accelerated 1110−/−mouse IBD model and analysis. At 8-12 weeks of age, male and female 1/10−/−mice (n=7 per sex) were switched to a diet containing 100 ppm of piroxicam (Sigma) (Envigo, TD.210442) to induce colitis development. Male (n=3) and female (n=4) 1/10−/−mice were maintained on the control diet for the duration of the experiment to serve as pre-IBD controls. Mice were euthanized at 18 days to collect tissues for assessment and intestinal contents for quantification of SoLs. At necropsy, IBD severity was first grossly assessed, which included qualitative evaluations of cecal atrophy (0-5), thickening of cecal (0-5) and colon tissues (0-5), extent of content loss in the cecum (0-4) and diarrhea (0-3). For histopathology, segments of the colon were first washed in PBS and then fixed in 10% neutral buffered formalin. The tissues were embedded in paraffin, cut into 5-mm sections, and stained with hematoxylin and eosin (H&E). Inflammation scores of colon sections were blindly assessed using an Echo Revolve light microscope and accompanying software. Briefly, IBD severity was assessed based on the following histopathological features: length measurements in microns of crypt hyperplasia converted to a score from 0-4, qualitative assessment of goblet cell loss (0-5), crypt abscesses per 10× field counts converted to a score from 0-4, and qualitative assessment of submucosal edema (0-3). RNA isolations and RT-qPCR were performed. Briefly, RNA was isolated from snap frozen colon tissues using the TriZol method (Thermo Fisher Scientific). cDNA was synthesized using SuperScript III reverse transcriptase (ThermoFisher Scientific). The relative abundance of mammalian mRNA transcripts was calculated using the delta delta CT method and normalized to Eef2 levels.

Fecal samples were collected and immediately flash frozen and stored at −80° C. For SoL extraction and quantification, frozen fecal samples were lyophilized to remove remaining water and subsequently resuspended in methanol (ThermoFisher Scientific). Fecal sample suspensions were vortexed for 1 minute prior to sonication for 10 minutes. The methanol extract was collected by centrifugation at 20,000×g for 10 minutes and dried under a gentle stream of nitrogen. The resulting residue was then redissolved in methanol+0.1% ammonium hydroxide (ThermoFisher Scientific) and filtered through a 0.22 μm filter prior to analysis. High-resolution mass spectra were collected using a ThermoFisher Scientific Q-Exactive HF-X hybrid Quadrupole-Orbitrap mass spectrometer using electrospray ionization in negative mode. Liquid chromatography used a ThermoFisher Scientific Vanquish HPLC coupled to the aforementioned mass spectrometer. Liquid chromatography (LC) was performed using a Waters Xbridge BEH C18 XP column (2.1×100 mm) with mobile phases A (water+0.1% ammonium hydroxide) and B (acetonitrile+0.1% ammonium hydroxide) in a gradient starting from 10% B and increasing to 100% B over 5 minutes, hold at 100% B for 2.5 minutes, then re-equilibration at 10% B for 2.5 minutes. MS scans were obtained in the orbitrap analyzer which was scanned from 500 to 2000 m/z at a resolution of 60,000 (at 200 m/z). MS data was analyzed by Thermo Xcalibur (4.2.47).

### Example 3

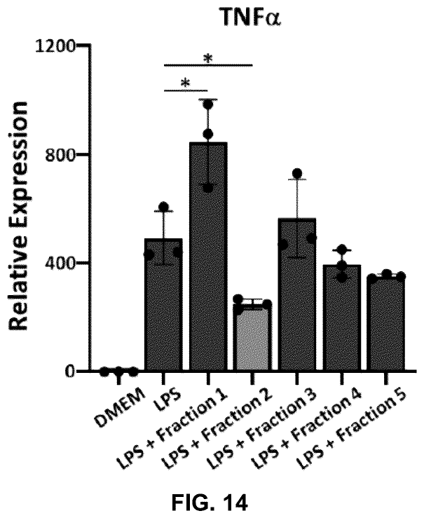

Crude extracts of the Alistipes and Odoribacter strains were fractionated and the biological activity of each fraction was determined using a cell-based assay that measured the suppression of LPS-induced TNFα production (FIG. 14). Briefly, a crude extract of A. timonensis DSM 27924 was separated based on polarity into 5 fractions. Each fraction was used in an in vitro cell-based assay to measure its respective capacity to suppress LPS-induced expression of TNFα. Fraction 2 was found to have the most significant anti-inflammatory effect compared to LPS. All fractions were compared to LPS for statistical significance with only fraction 1 and 2 showing significant change. Fractions 3, 4, and 5 showed no significant change. Statistical significance was determined using Student's t-test. Bars on the figures represent mean±standard error. For all p values: * 0.01<p<0.05.

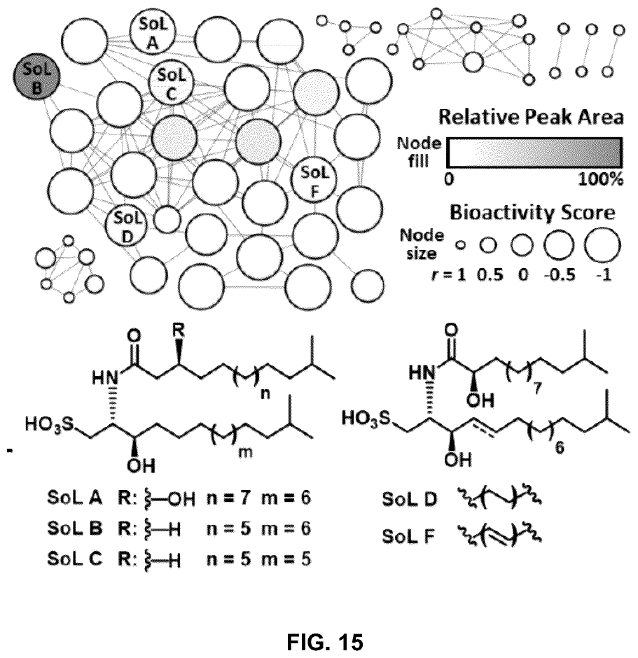

Each fraction was simultaneously analyzed by untargeted HPLC-HRMS/MS to generate molecular networks using the Global Natural Products Social (GNPS) feature-based molecular networking (FBMN) pipeline. The relative expression of TNFα in each fraction was then correlated with the relative peak area of molecular features across all fractions to generate a bioactivity score reflecting the contribution of specific features to the activity of the fractions. Bioactivity scores and relative peak areas were then mapped onto the molecular network to visualize these contributions. A representative bioactive molecular network generated from A. timonensis DSM 27924 is presented in FIG. 15. Specifically, FIG. 15 illustrates the relative peak area of each molecular feature in fraction 2 mapped to nodes with more abundant features increasing from white to darker shades. Bioactivity score was mapped to the node size with larger nodes indicating stronger negative correlations. Several known SoLs were annotated in this cluster and their structural variations are illustrated, further demonstrating that SoLs as a family of molecules contribute to the observed suppression of LPS-induced TNFα expression.

The fraction 2 SoL-containing cluster contained the most abundant and most active molecular features, as indicated by the node size and shade intensity of the map of FIG. 15 compared to other clusters in the network. Additionally, this cluster contained several known SoLs but many more unannotated SoLs, suggesting that the family of biologically active SoLs is larger than what is currently known. In all other SoL-producers tested, SoLs were consistently identified as a major contributor in the active fractions of each strain. To exclude the possibility of observed Sol activity being influenced by LPS contamination, the absence of leftover LPS in the Sol samples was confirmed using a chromogenic LAL assay. Narrowing down the immunosuppressive activity of each of the strains to SoLs was a guide to isolate pure SoLs A and B from A. timonensis DSM 27924 (structures confirmed by NMR spectroscopy), as well as from each of the other Alistipes and Odoribacter strains tested, the contribution of this class of lipids to the observed biological activity of Alistipes and Odoribacter was thus reinforced.

### Methods

Anaerobic culture and bioactive molecular networking. Three Alistipes and two Odoribacter strains were cultured in Reinforced Clostridial Medium (RCM, BD Biosciences) under anaerobic conditions at 37° C. After three days of growth, cultures were harvested by centrifugation at 12,000×g for 30 minutes. The resulting cell-free supernatant was extracted with an equal volume of methyl ethyl ketone and the cell pellets were extracted by resuspension in methanol and sonication before both extracts were combined and concentrated in vacuo. The combined crude extract was then fractionated on a silica gel column using a stepwise gradient of dichloromethane and methanol (DCM: MeOH; 15:1, 7:1, 5:1, 3:1, 1:1). Each fraction was then used in an in vitro cell-based assay measuring the suppression of LPS-induced TNFα expression. Simultaneously, samples of the fractions were subjected to untargeted HPLC-HRMS/MS. MS/MS was conducted using data-dependent acquisition with a resolution of 30,000, isolation window of 2.0 m/z, and dynamic exclusion time of 15 seconds. HPLC-HRMS/MS data was processed using MZmine3 following the GNPS FBMN workflow with minimal changes. Molecular networks were constructed using the quickstart GNPS FBMN setting with no changes. Bioactivity scores were assigned using a custom R script which calculated Pearson correlation coefficients between each molecular feature and the activity of each fraction. Finally, bioactive molecular networks were visualized in Cytoscape v3.9.1.

Purification of SoLs A and B. Fractions containing SoLs were further purified by Sephadex LH-20 run in 1:1 DCM: MeOH. Pure SoLs A and B were isolated by semi-preparative scale HPLC running an isocratic solvent composition of 47% water (with 0.1% ammonium hydroxide) and 53% acetonitrile (with 0.1% ammonium hydroxide) on a ThermoFisher Scientific Ultimate 3000 semi-preparative scale HPLC equipped with a Waters Xbridge Prep C18 5 μm OBD column (19×100 mm). ¹H, ¹³C, ¹H-¹³C HSQC, ¹H-¹³C HMBC, and ¹H-¹H COSY NMR spectra for SoLs A and B were acquired in methanol-d4 on a Bruker Avance III HD 400 MHz spectrometer with a 5 mm BBO ¹H/19F-BB-Z-Gradient prodigy cryoprobe. Data were collected and reported as follows: chemical shift, integration multiplicity (s, singlet; d, doublet; t, triplet; m, multiplet), coupling constant. Chemical shifts were reported using the methanol-d4 resonance as the internal standard for ¹H-NMR methanol-d4: δ=3.31 ppm and ¹³C-NMR methanol-d4: δ=49.0 ppm. Pure SoLs A and B were confirmed to be free of LPS using a Chromogenic Endotoxin Quant Kit (Pierce).

### Example 4

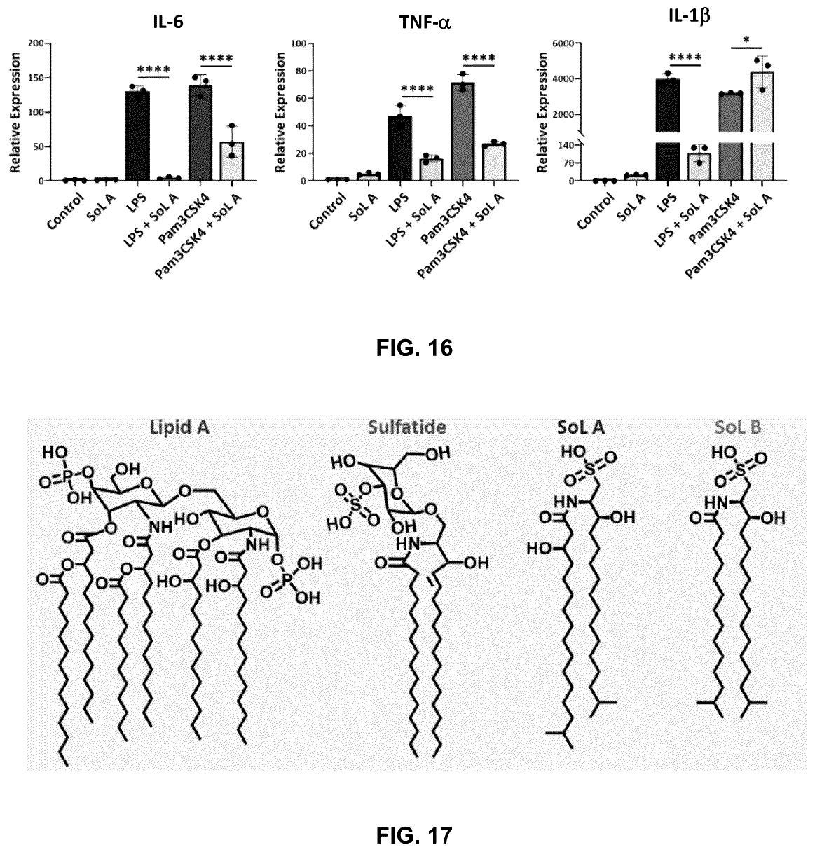

Primary mouse macrophages were treated with SoL A (as a representative of SoLs) either alone or together with LPS (an agonist of TLR4) or Pam3CSK4 (an agonist of TLR1/2) and the expression of three inflammatory cytokines was measured (IL-6, TNFα, and IL-1β). Results are shown in FIG. 16. By itself, SoL A exhibited a mild to moderate effect on the expression of pro-inflammatory cytokines compared to control. As expected, the TLR ligands, LPS and Pam3CSK4, both showed significant induction of all three cytokines compared to control (Student's t test, p≤0.0001). Notably, SoL A was found to significantly suppress the expression of all three cytokines induced by LPS (p≤0.0001).

SOL A also inhibited Pam3CSK4-induced IL-6 and TNFα to a smaller extent while increasing IL-1β expression induced by Pam3CSK4 (p≤0.05) (FIG. 16). On FIG. 16, bars represent mean±standard error. For each treatment, n=3. Significance was determined using Student's t test: * p<0.05, **** p<339 0.0001.

This result indicates that SOL A primarily affects LPS-induced inflammation and implies that interaction with TLR4 may be involved in SOL A's mechanism of action. Interestingly, SoL A's partial suppression of Pam3CSK4-induced inflammation suggests that SoL A-related anti-inflammatory activity may also extend to the TLR1/2 pathway, albeit to a lesser extent, and warrants further investigation.

### Methods

Preparation and treatment of macrophages. Primary mouse macrophages were prepared by first introducing 3 mL of 3% thioglycolate to mice via intraperitoneal injection. After 3 days, 10 mL of chilled PBS was introduced intraperitoneally to flush out macrophages. The cell suspension was then separated by centrifugation at 300×g for 5 minutes. Cells were seeded in culture dishes containing DMEM with 10% FBS for 1 hour before being washed with serum-free DMEM two times to remove unattached cells. The cells were incubated in serum-free DMEM for 16 hours before treatment. To treat the macrophages, cells were incubated for 6 to 24 hours in DMEM without FBS with addition of LPS (Sigma-Aldrich), Pam3CSK4 (Invivogen), or SoL A. Cells were finally washed twice with Dulbecco's phosphate-buffered saline before being lysed for total RNA or protein extraction.

mRNA extraction and RT-qPCR in macrophage-based assays. Treated mouse macrophage cells were lysed with TriZol (Invitrogen) and total RNA was extracted from the cell lysate using a Direct-zol RNA miniprep kit (Zymo Research) according to the manufacturer's protocol. The quality and quantity of RNA was then determined using a nanodrop and 1000 ng of mRNA from each sample was used for cDNA synthesis using a First-strand cDNA Synthesis System (Marligen Bioscience), qPCRs reactions were prepared in a 20 uL final volume containing Fast Start Universal SYBR Green Master (Rox) (Roche Applied Science), cDNA template, deionized water, and primers and probes for IL-1β, TNFα, IL-6, and the 18S rRNA which was used as a housekeeping gene Cycling conditions were 95° C. for 10 min followed by 40 cycles of 95° C. for 10 seconds, 60° C. for 15 seconds, and 68° C. for 20 seconds, then a melting curve analysis from 60° C. to 95° C. every 0.2° C. was obtained. Amplifications were performed on an Eppendorf Realplex Mastercycler (Eppendorf). Relative gene expression levels were calculated using the ΔΔCT method and expression levels of 18S were used to normalize the results.

### Example 5

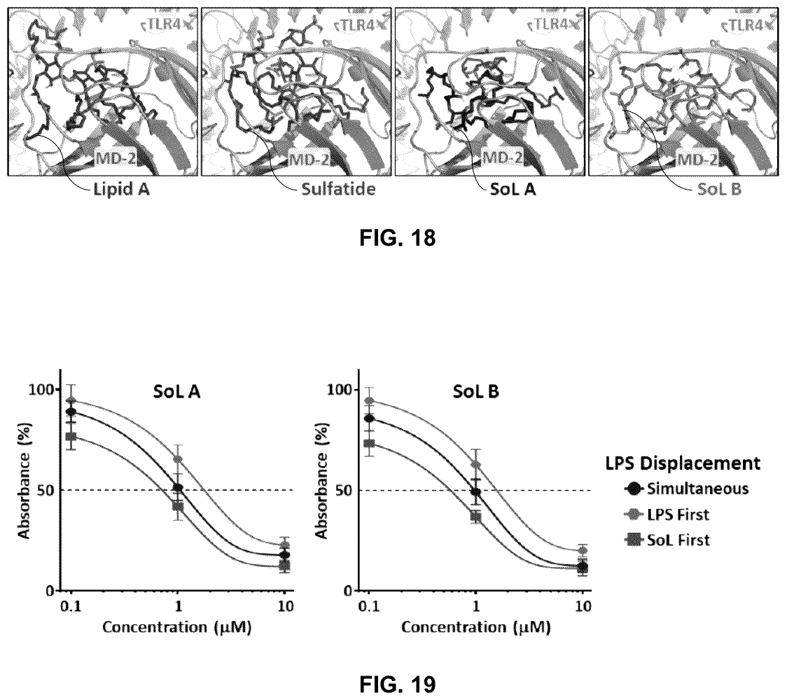

Comparing the chemical structure of SOL A to those of sulfatide and lipid A (the immunogenic portion of LPS) (FIG. 17), structural similarity was noted in the negatively charged head groups and multiple acyl chains. Inspired by sulfatides that bind in triplicate to MD-2, a molecular docking was used to model the binding of three molecules of SOL A to MD-2. The analysis predicted three molecules of SOL A indeed bind in the hydrophobic pocket of MD-2 (FIG. 18), where lipid A is known to bind, with a docking score of −8.9 kcal/mol, better than that of lipid A which had a docking score of −6.2 kcal/mol. Three molecules of each of SoLs A and B were used in molecular docking experiments to mimic the six acyl chains of lipid A as inspired by sulfatides. Additionally, SoL A was predicted to make hydrophobic contacts with several amino acids including 1117, F119, 152, and F121, all of which are also reported to contact the acyl chains of lipid A. Notably, SoL A is also predicted to contact residues including R264 and R90, consistent with contacts between these residues and the phosphate groups of lipid A. This suggests that SoL A may bind directly to the TLR4/MD-2 complex and possibly compete for binding with LPS, allowing it to suppress LPS-induced activation of the TLR4 pathway. A critical aspect of lipid A binding to MD-2 is the exclusion of one acyl chain from the hydrophobic pocket of MD-2 which forms a bridge with TLR4 and is involved in inducing dimerization. Likewise, it was observed that one acyl chain of SOL A was excluded from the hydrophobic pocket in the docking analysis (FIG. 18), further suggesting that SoL A mimics LPS as a ligand for TLR4. After successfully docking of SOL A, SoL B was tested. SoL B lacks an extra hydroxy group (FIG. 17) that may increase its interactions with the hydrophobic binding pocket of MD-2. The docking analysis showed that SoL B also binds to MD-2 (FIG. 18), with similar contacts as SoL A but higher affinity (docking score of −9.6 kcal/mol) as predicted.

To experimentally determine if SoLs A and B bind to MD-2 and to what extent the SoLs compete with LPS for binding to MD-2, an ELISA-based displacement assay was conducted. Taking advantage of biotinylated LPS, which retains the activity of unconjugated LPS, absorbance generated by an HRP-linked streptavidin probe was measured to determine the relative amount of MD-2 that was bound with biotin-LPS as opposed to MD-2 bound with SoL A or B. 0.1, 1.0, and 10 UM concentrations of SOL A or B and 1 ng/ml biotin-LPS were administered to MD-2 in three sequences: 1) Sol first followed by LPS 1 hour later, 2) LPS first followed by Sol 1 hour later, and 3) both SoL A or B and LPS at the same time. After 1 hour of incubation, it was found that at all concentrations, when SoL A or B was added first, there was a marked decrease in percent absorbance as compared to when LPS was added first and when the two compounds were added together (FIG. 19). Results suggests that SoLs A and B both bind and occupy some sites of MD-2, preventing LPS from fully binding when it is added 1 hour after SoL A or B. Furthermore, when moving from low to high concentrations of SOL A or B, it was observed that the percent absorbance decreased dramatically. This indicates that with increasing concentration of Sol A or B, less LPS binds to MD-2, implying that SoLs indeed compete with LPS for binding to MD-2. Taken together, these results indicate that SoLs A and B can bind directly to MD-2 and more importantly compete with LPS for binding to this target, thus providing a potential molecular mechanism underlying SoL A's pro-inflammatory activity by itself as well as its strong activity in suppressing LPS-induced inflammation which likely also expands to other members of SoLs family.

### Methods

Molecular docking. The crystal structure of the TLR4/MD-2 complex was retrieved from the Protein Data Bank (PDB ID: 3FXI) and prepared using AutoDock Tools. Molecular structures of SOL A, SoL B, sulfatide, and lipid A were constructed, and energy minimized using Marvin version 21.17.0, ChemAxon. Models of SOL A, SoL B, sulfatide, and lipid A were also prepared using AutoDock Tools and docked against the TLR4/MD-2 complex using AutoDock Vina in a 32×32×32 angstrom box surrounding the MD-2 monomer. Docking results were visualized using PyMol.

ELISA displacement assay. Solid-phase sandwich ELISA kits were purchased from Invitrogen. The ELISA experiments were performed according to the kit instructions, using 50 nM hMD-2 (Novus Biologicals), 1 ng/mL LPS-EB Biotin (Invivogen), and 0.1, 1.0, and 10 UM purified SoL A. SOL A was added to the assay 1 hour before, 1 hour after, or simultaneously with LPS-EB Biotin. Absorbance was measured at 450 nm using a BioTek microplate reader.

### Example 6

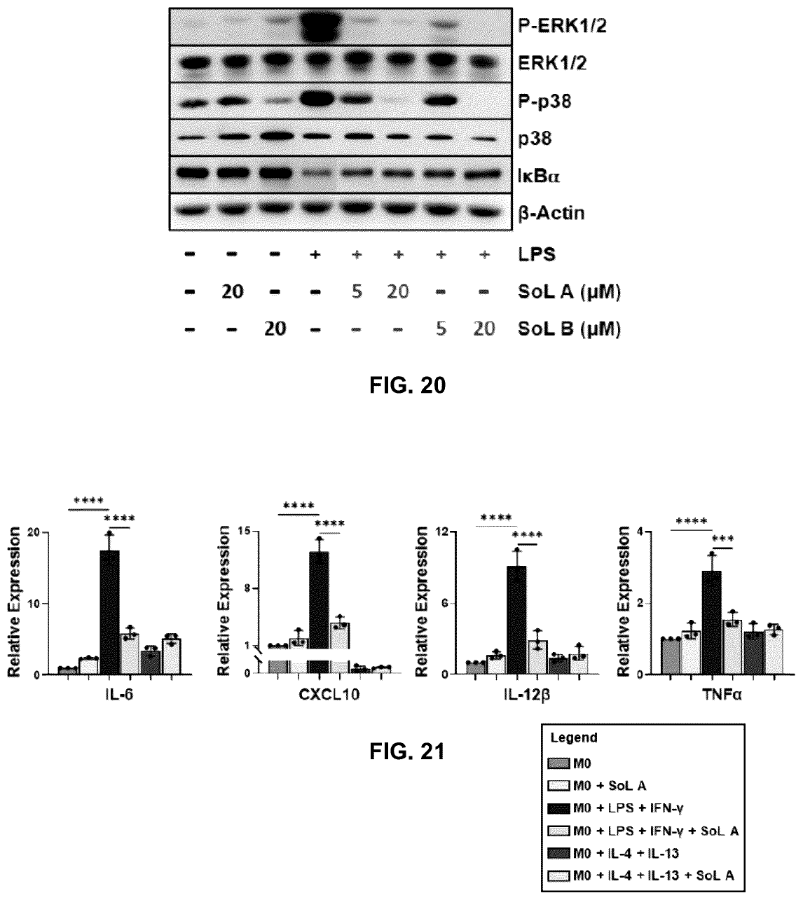

Macrophages were treated with 100 ng/ml LPS in the presence of increasing concentrations of Sol A or B (from 0 to 20 μM) for 30 minutes. Following treatment, all cellular protein was extracted using MPER lysis buffer (Thermo Scientific). Protein samples were loaded onto SDS-PAGE gels for separation, then transferred to nitrocellulose membranes (Amersham Biosciences). Primary antibodies and HRP-conjugated secondary antibodies (Cell Signaling Technology) were used to detect target proteins. Signal was detected using an ECL kit (Thermo Scientific). The Western blot analysis (FIG. 20) found that both SoLs reduced LPS-induced phosphorylation of p38 and ERK1/2 in a concentration-dependent manner. At the concentration of 20 UM, SoLs A and B almost completely blocked LPS-induced phosphorylation of p38 and ERK1/2. The Western blot also showed that SoLs concentration-dependently suppressed LPS-induced IκBα degradation. The housekeeping gene β-Actin was used as a loading control.

### Example 7

Because TLR4 signaling leads to macrophage polarization which has been shown to contribute to IBD, the effects of SOL A on macrophage polarization were examined. THP-1 monocytes were treated with IFN-γ and LPS to induce M1 polarization or IL-4 and IL-1β to induce M2 polarization. Successful induction of M1 and M2 polarization was confirmed by morphology changes and subsequent RT-qPCR quantification of cytokine profiles.

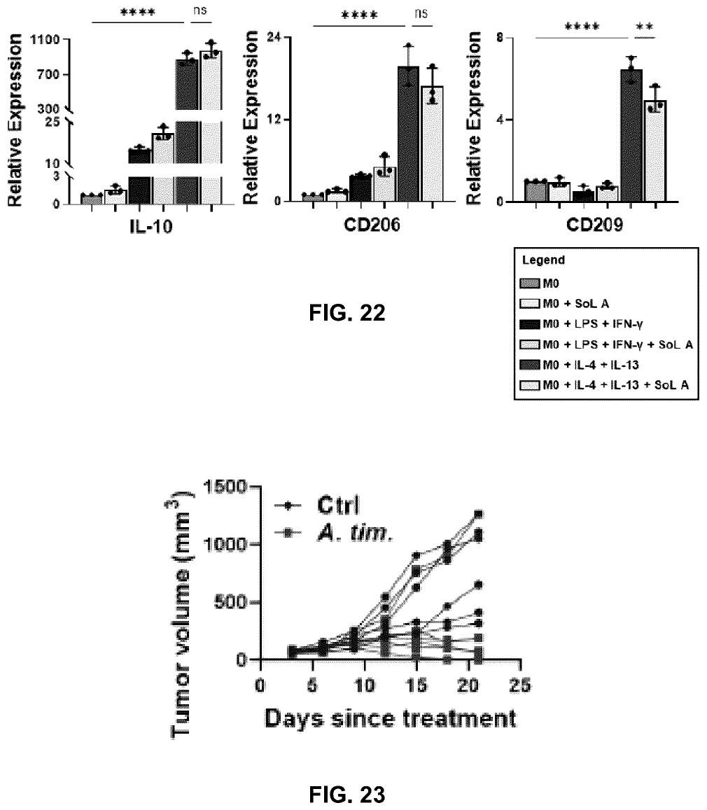

When 10 UM of SOL A was added alongside the respective inducing agents, the relative cytokine expression results showed that SoL A significantly reduced the production of M1-polarized macrophage markers IL-6, CXCL10, IL-12β, and TNFα, compared to macrophages treated without SoL A (FIG. 21) but had a mostly non-significant effect on M2 polarization (FIG. 22). This suggested that SoL A suppresses macrophage M1 polarization, which supports the aforementioned result that SoLs interfere with TLR4 signaling potentially leading to inhibition of TLR4-mediated IBD. For all treatments, n=3 and significance was determined using one-way ANOVA: 0.001<p<0.01, *** 0.0001<p<0.001, and **** p<0.0001. These results support that SoLs exert their anti-inflammatory effect by blocking LPS-mediated phosphorylation of downstream TLR4 proteins, effectively negating LPS activation of the TLR4 pathway.

THP-1 monocytes were maintained in RPMI16 with 10% heat inactivated FBS, 1% Penicillin-Streptomycin-Ampotericin B, and 50 μM 2-mercaptoethanol prior to differentiation. The cells were differentiated into macrophages with 150 nM PMA for 48 hours. M1 polarization was induced by adding 20 ng/ml IFN-γ and 100 μg/mL LPS for 24 hours. M2 polarization was induced by adding 20 ng/ml IL-4 and 20 ng/ml IL-1β for 24 hours. In all tests, 10 UM Sol A was added at the same time as M1 and M2 differentiation agents. After 24 hours of treatment, total RNA was collected, and RT-qPCR was performed as described above.

### Example 8

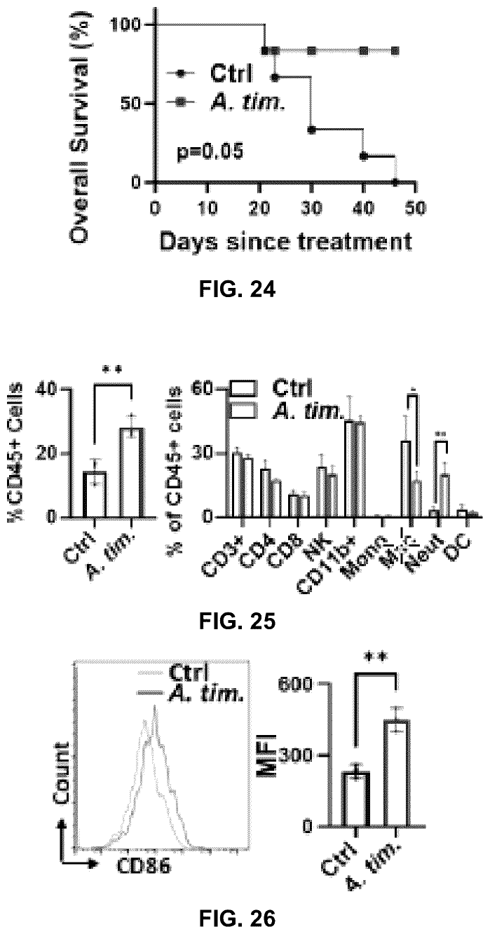

To explore the impact of gut Alistipes on breast cancer, intestines of FVB/N mice were colonized with A. timonensis followed by syngeneic transplantation with H605 tumor cells. Results showed that A. timonensis colonization inhibited tumor growth and improved overall survival rate (FIG. 23, FIG. 24). FIG. 23 presents growth kinetics of the H605 cells in the FVB/N mice. Antibiotics-treated mice were treated by oral gavage of 1×10⁸ CFU/mL A. timonensis or vehicle (Ctrl) twice before and once a week after tumor cell injection. FIG. 24 presents survival curves of the H605 tumor-bearing mice. Mice were euthanized once tumors reached 1,500 mm³ or at the end of experiments.

To test whether A. timonensis colonization modulates anti-tumor immune response, the immune profiles in tumor microenvironment were analyzed. In comparison to sham-treated mice, colonized mice tumors showed more immune infiltrates (CD45+ cells) among which the distribution of myeloid cells is skewed toward neutrophils and away from macrophages in the colonized mice (FIG. 25). Macrophage cells also tended to express higher levels of co-stimulatory molecule CD86 (FIG. 26, showing FACS analysis of CD86 expression in the gated macrophages (CD45+CD11b+Ly6G-Ly6C-F4/80+).

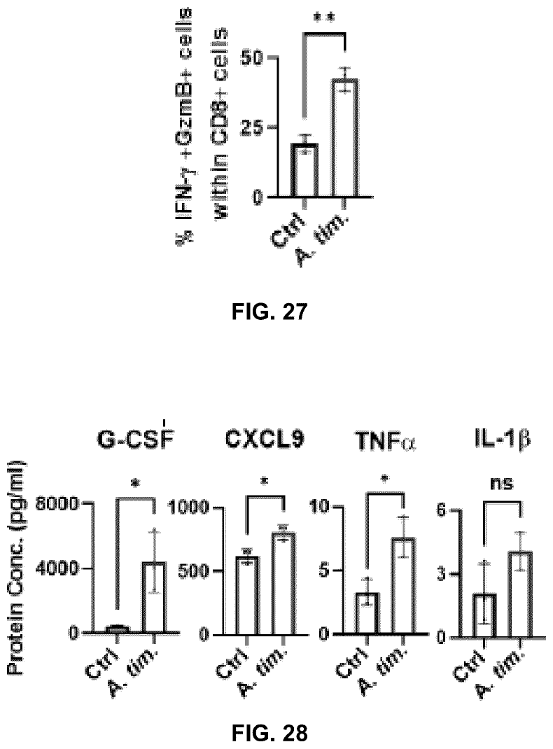

Although the relative frequencies of T cells were not significantly altered, there were higher frequencies of CD8+T cells producing the cytotoxic molecules IFN-γ and GzmB, indicating an active TME (FIG. 27).

To link gut microbial inoculation to the changes in TME, serum cytokines levels that reflect systemic immune response were measured. Consistently, significant increases were observed in the protein levels of G-CSF (neutrophil stimulation), CXCL9 (T cells recruitment), TNF-α (myeloid cells activation) and a slight increase in IL-1β expression (inflammation) (FIG. 28). Serum cytokine expression levels were measured using a 32-Plex assay.

Data are presented as mean±SE. Ctrl: vehicle-treated; A. tim.: A. timonensis colonized mice. *, p<0.05; **, p<0.01. ns, not significant. The expression of other common chronic cytokines such as IL-6, IL10 and IL-1a were either not detectable or unchanged.

Overall, these results indicated that A. timonensis colonization enhances anti-tumor immunity rather than a typical chronic inflammation environment.

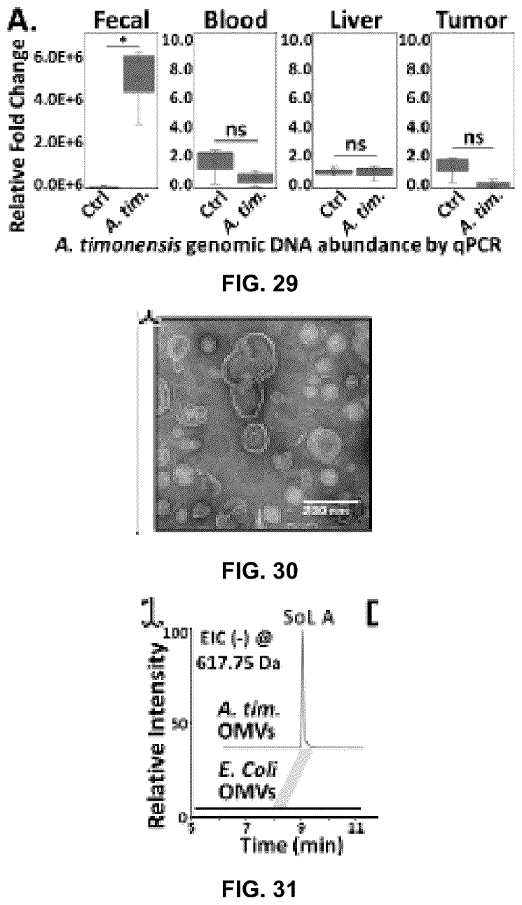

To test whether A. timonensis colonization triggers local and systemic inflammation through translocation, quantitative PCR was performed to detect A. timonensis genomic DNA (gDNA) in feces, liver, blood, and tumor samples. A high abundance of Alistipes gDNA was detected only in the feces of colonized mice, but not in other samples (FIG. 29), suggesting that bacterial translocation had not occurred and thus cannot account for the observed immune responses in A. timonensis colonized mice. It was thus hypothesized that Alistipes modulates the immune system through secreted metabolites. In support of this hypothesis, significantly increased Alistipes-derived SoLs was detected in the plasma of A. timonensis colonized mice.

To further explore this hypothesis, outer membrane vesicles (OMVs) were collected from the supernatant of A. timonensis culture (FIG. 30). Metabolic analysis (FIG. 31) showed that SoLs A and B were major components of the A. timonensis OMVs.

### Example 9

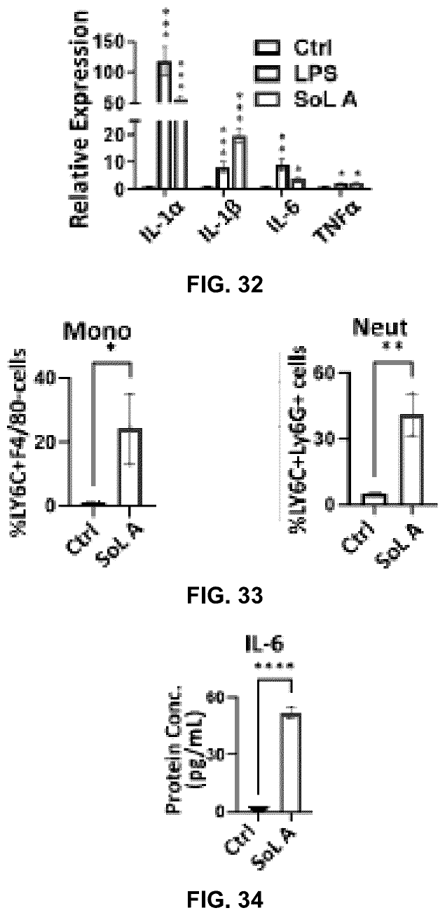

Considering that SoLs are potential PAMPs, tests were carried out to determine whether SoL A, as a representative SoL, can induce inflammatory cytokine expression by treating peritoneal macrophages with purified SoL A. As indicated in FIG. 32, the expression levels of several pro-inflammatory cytokines including IL-1a, IL-1B, IL-6, and TNFα were significantly increased, displaying a similar trend to that induced by lipopolysaccharide (LPS).

### Example 10

To test whether SoL A can induce inflammation in vivo, mice were treated by intraperitoneal (i.p.) injection of 1 mg/kg of SOL A. At 2 hrs post-injection, FACS analysis of immune cells in the peritoneal exudate was performed. As indicated in FIG. 33, more monocytes (Mono) and neutrophils (Neut) were recruited into the peritoneal cavity, indicating that local inflammation was triggered. In addition, serum IL-6 levels were significantly and consistently increased in the Sol A-injected mice (FIG. 34). These results thus demonstrate that SoL A by itself can induce inflammatory responses.

While certain embodiments of the disclosed subject matter have been described using specific terms, such description is for illustrative purposes only, and it is to be understood that changes and variations may be made without departing from the spirit or scope of the subject matter.

What is claimed is:

1. A method for regulating toll-like receptor 4 signaling in an immune cell comprising delivering a sulfonolipid to the immune cell, wherein upon the delivery, signaling of the toll-like receptor 4 is suppressed.

2. The method of claim 1, wherein the immune cell is a macrophage.

3. The method of claim 1, wherein the sulfonolipid is an Alistipes derived sulfonolipid.

4. The method of claim 1, wherein the sulfonolipid is a sulfobacin.

5. The method of claim 4, wherein the sulfobacin comprises sulfobacin A or sulfobacin B.

6. The method of claim 1, wherein the sulfonolipid is delivered in conjunction with a bacterium that produces the sulfonolipid as a metabolite.

7. The method of claim 1, wherein the method is an in vivo method.

8. The method of claim 7, wherein the in vivo method is in treatment of a subject diagnosed with an inflammatory bowel disease, atherosclerosis, or a cancer.

9. The method of claim 8, wherein the cancer is a breast cancer, a melanoma, or a lung cancer.

10. A composition comprising a sulfonolipid and a pharmaceutically acceptable carrier.

11. A method for determining a correlation between a gut microbial metabolite and a disease, comprising:
identifying a reference gene sequence, wherein the reference gene sequence comprises the sequence of an enzyme utilized in microbial derivation of the metabolite;
identifying a set of homologous gene sequences in a reference genome that exhibit a sequence similarity of about 50% or greater to the reference gene sequence;
classifying the homologous gene sequences in the set according to genera and/or bacterial families;
dividing each classification to form prioritized subfamilies, wherein the members of each prioritized subfamily exhibits a sequence similarity to one another of about 90% or greater;
comparing the abundance the prioritized subfamilies in a first metagenomic dataset of a healthy cohort with those in a second metagenomic dataset of a disease cohort; and
comparing the abundance of a transcript of the prioritized subfamilies in a first metatranscriptomic dataset of a healthy cohort with those in a second metatranscriptomic dataset of a disease cohort; wherein
a statistically significant difference in the presence of the prioritized subfamilies in the first healthy cohort and the first disease cohort and in the transcripts of the prioritized subfamilies in the second healthy cohort and the second disease cohort indicates a correlation between the gut microbial metabolite and the disease.

12. The method of claim 11, wherein the members of each prioritized subfamily includes at least one similar protein domain.

13. The method of claim 11, the method including identifying one or more additional reference gene sequences, wherein the one or more additional reference gene sequences comprise sequences of one or more additional enzymes utilized in microbial derivation of the metabolite.

14. The method of claim 13, wherein the members of each prioritized subfamily each include all of the one or more additional reference gene sequences.

15. The method of claim 11, further comprising selecting a representative member or members of each prioritized subfamily for the comparisons.

16. The method of claim 15, wherein only a single member of each prioritized subfamily is selected.

17. The method of claim 11, wherein the first metagenomic dataset of the healthy cohort and the first metatranscriptomic dataset of the healthy cohort are obtained from a single dataset.

18. The method of claim 11, wherein the first metagenomic dataset of the disease cohort and the first metatranscriptomic dataset of the disease cohort are obtained from a single dataset.

19. The method of claim 11, further comprising comparing the abundance of the metabolite in a first metabolomic dataset of a healthy cohort with those in a second metabolomic dataset of a disease cohort.

20. The method of claim 11, wherein the gut microbial metabolite is a sulfonolipid.
